(19) United States
(12) Plant Patent Application Publication
(10) Pub. No.: US 2014/0259241 P1
(43) Pub. Date: Sep. 11, 2014

(54) Phlox paniculata plant named 'Green Lady'

(50) ;

(71) Applicant: Efraim Gafni, Kfar Hess (IL)

(72) Inventor: Efraim Gafni, Kfar Hess (IL)

(21) Appl. No.: 13/815,495

(22) Filed: Mar. 7, 2013

(51) Int. Cl. A01H 5/00

(52) U.S. Cl. PLT/320

(57) ABSTRACT

A Phlox paniculata plant particularly distinguished by inflorescences atypical of Phlox flowers due to total absence of petals, inflorescences of 50-80 cm tall producing clusters of green buds and absence of reproductive organs, is described.

### GENUS AND SPECIES

Phlox paniculata

### VARIETY DENOMINATION

‘Green Lady’

### BACKGROUND OF THE INVENTION

The present invention relates to a new and distinct cultivar of Phlox paniculata hereinafter referred to by the cultivar name ‘Green Lady’ or the ‘new plant’. ‘Green Lady’ was discovered by the inventor in August 2007 as a naturally occurring single whole plant mutation within a population of Phlox paniculata ‘Amethyst’ (unpatented) propagated from stem cuttings in a nursery located in Kfar Hess, Israel.

The new plant has been asexually propagated by stem cuttings at the same nursery and the unique characteristics of the new plant have been found to be reproducible and stable in successive generations of asexually reproduced plants and the resultant plants have been found to be identical to the original selection.

Plant Breeders rights application for ‘Green Lady’ was filed in Israel on 25 Jun. 2012 and assigned application number 4395/12. Plant Breeders rights application for ‘Green Lady’ was filed in Europe on 24 Jan. 2013 and assigned application number 2013/0113. ‘Green Lady’ has not been sold or made publicly available more than one year prior to the filing of this application.

### SUMMARY OF THE INVENTION

The following are the most outstanding and distinguishing characteristics of the new plant when grown under normal horticultural practices in Kfar Hess, Israel:
1. Inflorescences atypical of Phlox flowers due to total absence of petals
2. Inflorescences of 50-80 cm tall producing clusters of green buds
3. Absence of reproductive organs

### DESCRIPTION OF THE PHOTOGRAPHS

The new plant is illustrated by the accompanying photographs which show the plant habit, mature inflorescence and foliage of the plant. The colors shown are as true as can be reasonably obtained by conventional photographic procedures. The photographs are of a plant approximately 24 months old, after planting in optimum conditions in Kfar Hess, Israel.

FIG. 1 shows a close-up of the mature inflorescence.

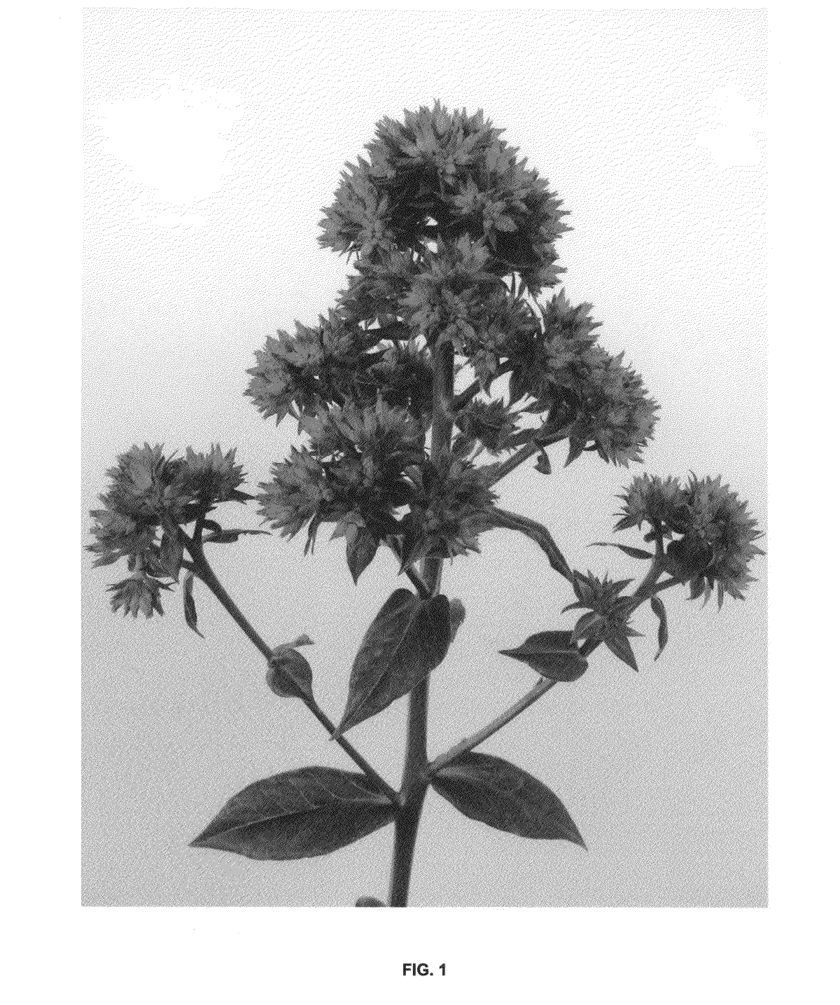

FIG. 2 shows a close-up of the leaf

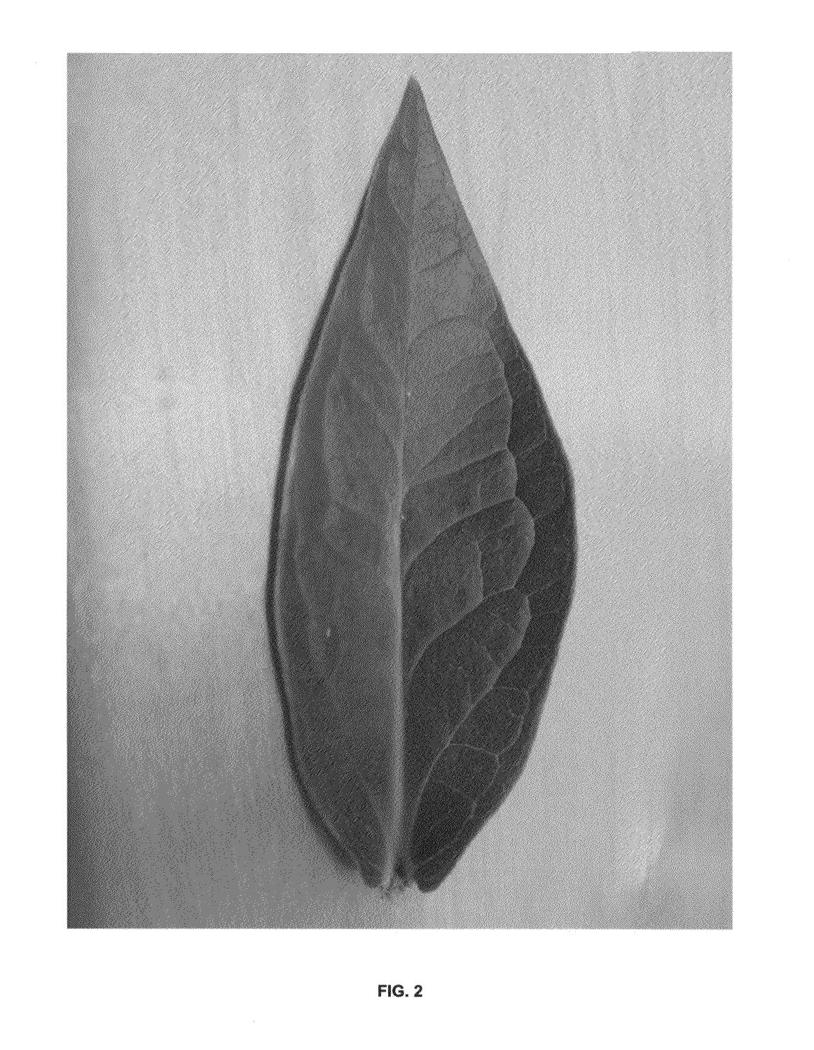

FIG. 3 shows the mature inflorescence and foliage.

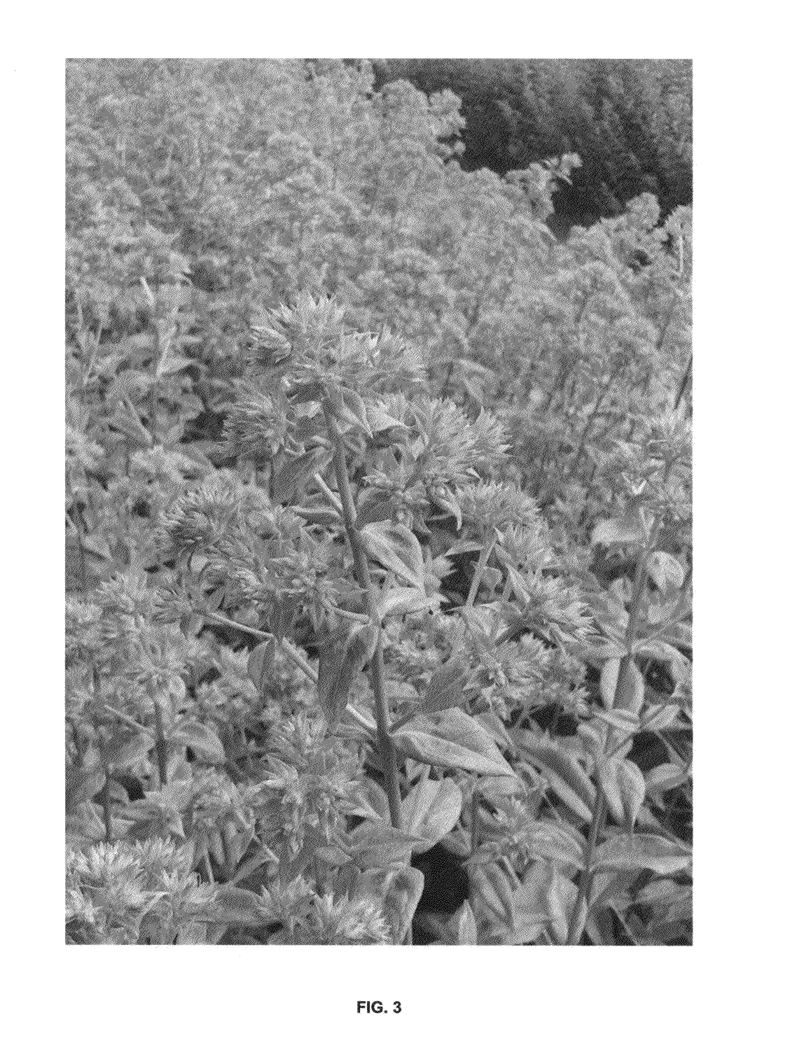

### DESCRIPTION OF THE NEW CULTIVAR

The following detailed description sets forth the distinctive characteristics of ‘Green Lady’. The following observations, measurements and values describe the new cultivar at 24 months of age as grown in Kfar Hess, Israel, under conditions which closely approximate those generally used in commercial practice. Colour readings were taken under natural light. Color references are primarily to the R.H.S. Colour Chart of The Royal Horticultural Society of London (R.H.S.), except where general colors of ordinary significance are used.

### DETAILED BOTANICAL DESCRIPTION

Classification:
   Family.—Polemoniaceae.
   Botanical name.—Phlox paniculata.
   Common name.—Phlox.
Parentage: Non-induced single whole plant mutation of Phlox paniculata ‘Amethyst’ (unpatented).
Plant:
   Plant habit.—Upright herbaceous evergreen perennial.
   Growth habit.—Moderate vigour, half-hardy and freely branching.
   Height (measured from top of the soil).—50 cm to 100 cm.
   Diameter or area of spread.—20 cm to 30 cm.
   Propagation.—Vegetative cuttings or bare root division.
   Time to initiate roots.—18 days (summer); 30 days (winter).
   Time to produce a rooted liner (young plant).—30 days (summer); 45 days (winter).
   Time to produce a flowering plant from rooting cutting.—80-180 weeks.
   Rooting habit.—primary roots up to 1 mm diameter and secondary fibrous and freely branching.
   Root colour.—RHS 159A.
Stems:
   Stem length.—40 cm to 90 cm.
   Stem diameter.—5 mm to 15 mm.
Branches:
   Number of lateral branches per plant.—Up to 20.
   Lateral branch length.—5 cm to 20 cm.
   Lateral branch diameter.—3 mm to 5 mm.
Leaves:
   Arrangement.—Opposite.
   Color.—Young leaves upper surface: RHS 146A Young leaves lower surface: RHS 146B Mature leaves upper surface: RHS137A Mature leaves lower surface: RHS138A.
   Length.—4 cm to 9 cm.
   Width.—2 cm to 4.5 cm.
   Shape.—Lanceolate.
   Apex.—Broadly acuminate.
   Base.—Attenuate sessile base.
   Margin.—Entire, minutely pubescent.
   Texture.—Smooth and glabrous on both sides, no pubescence.
   Quantity.—20 to 40 leaves per main branch.
   Venation pattern.—Arcuate.
   Venation color (upper surface).—RHS11D.
   Venation color (lower surface).—RHS160A.
   Petioles (leaf stem).—Leaves are sessile.
Peduncle (flower stem):
   Quantity per plant.—20 to 100.
   Angle.—main pedicle 0 degrees, lateral pedicle 55 degrees.
   Length.—Up to 5 cm.
   Diameter.—1.5 mm to 3 mm.
   Shape (cross section).—round.
   Color.—RHS146C.
Pedicel:
   Length.—1 mm to 3 mm.
   Diameter.—0.5 mm to 1 mm.
   Colour.—RHS145B.
Inflorescence:
   Inflorescence type.—terminal panicle.
   Blooming habit.—abundant clusters of flower buds. Continuous production in Israeli conditions with an average of 2.5 harvestable stems per year.
   Cut flower longevity.—10 to 21 days.
   Height.—5 cm to 15 cm.
   Width.—5 cm to 15 cm.
   Flower bud numbers per inflorescence.—50 to 250.
   Natural flowering season.—Summer.
   Fragrance.—Absent.
Flowers:
   Size of flower bud.—3 mm to 7 mm.
   Colour.—RHS146B.
   Flower type and habit.—Typical Phlox flowers do not develop. Panicles with numerous clusters of buds are present but petals and reproductive organs are absent.
Sepals:
   Texture.—Smooth and glabrous on both sides, no pubescence.
   Number.—5.
   Shape.—Lanceolate.
   Margin.—Entire.
   Apex.—Narrowly acute.
   Base.—Fused into a campanulate structure.
   Length.—3 mm to 5 mm.
   Width.—1 mm to 2 mm.
   Color.—RHS146B.
Reproductive organs: The flower buds have no reproductive organs.
Seeds: No fruit or seeds are developed.
Disease resistance/susceptibility: No observations made.
Insect resistance/susceptibility: No observations made

### Comparison with Parental and Known Cultivars

When compared to the commercial variety ‘Green Forest’ (unpatented); ‘Green Lady’ has a taller plant height, a faster growth rate and lighter green inflorescences which are more pyramidal in shape.

When compared to the commercial variety ‘Pleasant Feelings’ (U.S. Plant Pat. No. 16,596); ‘Green Lady’ has a slower growth rate, stronger peduncles (flower stems), straighter branches and inflorescences which are more pyramidal in shape.

When compared to the parent sport ‘Amethyst’ (unpatented); ‘Green Lady’ does not have petals or reproductive organs, and has a slower growth rate, stronger peduncles (flower stems), wider and shorter leaves, no variation in leaf color and larger inflorescences.

I claim:

1. A new and distinct cultivar of Phlox paniculata plant as shown and described herein.
